(19) United States
(12) Patent Application Publication
(10) Pub. No.: US 2021/0299432 A1
(43) Pub. Date: Sep. 30, 2021

(54) CENTRIFUGAL BLOOD PUMP

(71) Applicant: SOGANG UNIVERSITY RESEARCH AND BUSINESS DEVELOPMENT FOUNDATION, Seoul (KR)

(72) Inventors: Nahmkeon HUR, Seoul (KR); Seongwon KANG, Seoul (KR); Wonjung KIM, Bucheon-si, Gyeonggi-do (KR)

(73) Assignee: SOGANG UNIVERSITY RESEARCH AND BUSINESS DEVELOPMENT FOUNDATION, Seoul (KR)

(21) Appl. No.: 17/260,925

(22) PCT Filed: Jul. 2, 2019
(86) PCT No.: PCT/KR2019/008001

§ 371 Date: Jan. 15, 2021

(30) Foreign Application Priority Data
Jul. 17, 2018 (KR) 10-2018-0082947

(51) Int. Cl. A61M 60/419; A61M 60/216; A61M 60/806; A61M 60/122

(52) U.S. Cl. CPC A61M 60/419; A61M 60/216; A61M 2205/103; A61M 60/122; A61M 60/806

(57) ABSTRACT

A centrifugal blood pump includes: a housing; a suction inlet for introducing blood into the housing; an impeller that is rotatably disposed in the housing and imparts a centrifugal flow to the blood introduced through the suction inlet by rotation; and a discharge outlet for discharging the blood given a centrifugal flow by the impeller. The impeller is formed in a double impeller structure including double vanes arranged vertically.

### TECHNICAL FIELD

Centrifugal blood pumps that circulate blood using centrifugal force are used. Typically, a centrifugal blood pump includes an impeller for inducing a centrifugal flow of blood. The impeller typically has a conical shape and is disposed within a cone-shaped housing. The impeller is rotatably installed in the housing, and generally, the impeller may be configured to rotate by a magnet installed thereon rotating by a magnetic field formed by an external electromagnetic coil or may be configured to rotate by an external rotating magnet.

In addition, the centrifugal blood pump includes a suction inlet for introducing blood into the housing and a discharge outlet for discharging blood from the housing. Typically, the suction inlet is provided near the upper apex of the cone-shaped housing, and the discharge outlet is provided at the lower side of the housing. The blood introduced through the suction inlet has a centrifugal flow by the impeller and is then discharged through the discharge outlet. In this case, tubes through which blood flow may be connected to the suction inlet and the discharge outlet, respectively.

Since such a centrifugal blood pump has an operation way in which a centrifugal flow of blood is caused by the rotation of the impeller, hemolysis due to the destruction of blood is essential. Since it becomes a problem when there are a lot of hemolysis, there is a need to reduce the hemolysis phenomenon in a centrifugal blood pump.

prior document: U.S. Pat. No. 6,183,220 (Feb. 6, 2001)

### DETAILED DESCRIPTION OF THE INVENTION

### Technical Problem

The problem to be solved by the present invention is to provide a centrifugal blood pump having a structure that can minimize the hemolysis phenomenon.

### Technical Solution

A centrifugal blood pump according to an embodiment of the present invention includes: a housing; a suction inlet for introducing blood into the housing; an impeller that is rotatably disposed in the housing and imparts a centrifugal flow to the blood introduced through the suction inlet by rotation; and a discharge outlet for discharging the blood given a centrifugal flow by the impeller. The impeller is formed in a double impeller structure including double vanes arranged vertically.

The impeller may include: a base; a shroud that is formed to be upwardly spaced apart from an upper surface of the base; a shrouded vane extending between the upper surface of the base and a lower surface of the shroud; and an open vane formed on an upper surface of the shroud.

The shroud may include an inlet hole that is formed on a center region thereof, and the shrouded vane and the open vane may be not formed on a center region of the base.

The radially inner end of the shrouded vane may be formed to be inclined such that an upper end thereof is located radially outside from a lower end thereof.

An inner end of the shrouded vane may be not covered by the shroud, and an inclined line of the inner end of the shrouded vane may be connected to an inner upper end of the open vane.

An outer end of the shrouded vane may be formed to be vertical, and an outer end of the open vane may be formed such that a downstream side thereof in a rotation direction is inclined with respect to a vertical direction.

A ratio of a height of the outer end of the open vane and a height of the outer end of the shrouded vane may be between 1:1 and 1:2.

### Advantageous Effects

According to the present invention, by forming an impeller in a double vane structure including a shrouded vane and an open vane, it is possible to substantially reduce the hemolysis that may occur during the blood pumping process.

### BRIEF DESCRIPTION OF DRAWINGS

FIG. 1 is a perspective view of a centrifugal blood pump according to an embodiment of the present invention.

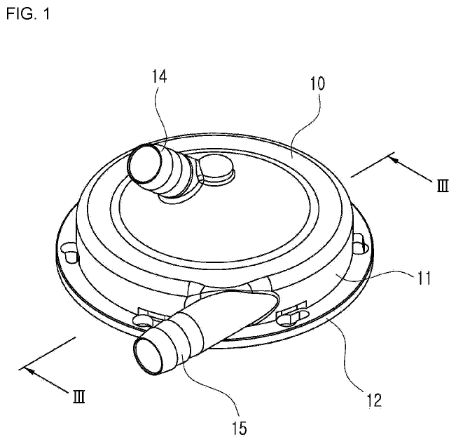

FIG. 2 is an exploded perspective view of a centrifugal blood pump according to an embodiment of the present invention.

FIG. 3 is a cross-sectional view taken along a line III-Ill of FIG. 1.

FIG. 4 is a top plan view of an impeller of a centrifugal blood pump according to an embodiment of the present invention.

### DETAILED DESCRIPTION OF THE EMBODIMENTS

An embodiment of the present invention will be described in detail with reference to the accompanying drawings.

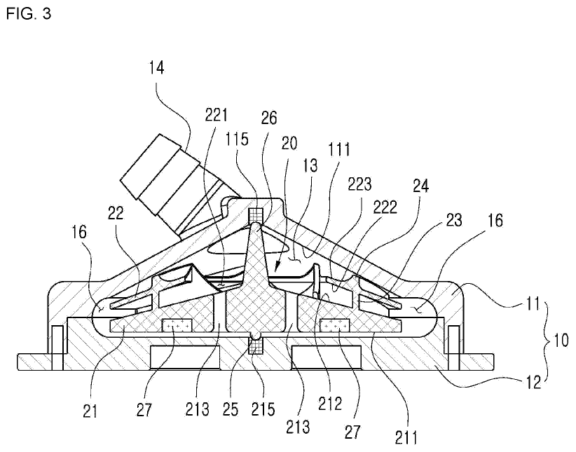

A housing 10 may include an upper housing member 11 and a lower housing member 12, and the upper and lower housing members 11 and 12 are fastened to each other to form a blood pumping space 13. As shown in FIG. 3, the blood pumping space 13 may have a cone shape with a pointed top. The upper and lower housing members 11 and 12 may be fastened to each other to form and may be fastened to each other by fastening members such as bolts.

A suction inlet 14 and a discharge outlet 15 are provided in the housing 10. As shown in the drawing, the suction inlet 14 may be provided on the upper portion of the housing 10, and the discharge outlet 15 may be provided on the side of the housing 10. Blood is introduced into the blood pumping space 13 in the housing 10 through the suction inlet 14 and is then discharged to the outside through the discharge outlet 15. Tubes (not shown) through which blood flows may be connected to the suction inlet 14 and the discharge inlet 15, respectively, so that blood can be introduced and discharged.

An impeller 20 is disposed rotatably within the housing 10. That is, the impeller 20 is rotatably disposed in the blood pumping space 13 of the housing 10. The impeller 20 is configured to impart a centrifugal flow to the blood introduced by rotation. For example, a magnet 27 may be embedded in the impeller 20, and the magnet 27 and the impeller 20 may be rotated by magnetic attraction using an electromagnetic coil disposed outside or a magnet installed in a motor.

At this time, as shown in the drawing, the suction inlet 14 may be inclined at a predetermined angle with respect to the upward direction of the impeller 20. The inclined suction inlet 14 may make the blood pressure uniform inside the pump and may reduce the hemolysis phenomenon.

The impeller 20 is formed in a double vane structure including two vanes 23 and 24 that are arranged vertically.

The impeller 20 may have a base 21 having a cone shape. A bottom surface 211 of the base 21 may be formed substantially flat to correspond to the shape of a lower inner peripheral surface 121 of the housing 10, and an upper surface 212 of the base 21 may be formed to have a shape a center portion of which protruded upward to correspond to the shape of an upper inner peripheral surface 111. Thus, the base 21 may have a cone shape that is pointed upward.

The impeller 20 includes a shroud 22 that is disposed to be spaced apart from the upper surface 212 of the base 21. A blood inlet hole 221 through which blood flows is provided at the center of the shroud 22. The shroud 22 may have an inclined shape in which the central portion thereof protrudes upward, and the blood inlet hole 221 may be formed at the central portion of the shroud 22. The blood inlet hole 221 may be disposed directly below the suction inlet 14, and blood introduced through the suction inlet 14 flows into the impeller 20 through the blood inlet hole 221.

A plurality of shrouded vanes 23 respectively connecting the upper surface 212 of the base 21 and a lower surface 222 of the shroud 22 are provided. Thereby, the blood flowing into the blood inlet hole 221 passes through a space between the shroud vanes 23 and is discharged through the outer edge between the base 21 and the shroud 22.

Meanwhile, a plurality of open vanes 24 are provided on an upper surface 223 of the shroud 22. The open vane 24 is formed by protruding from the upper surface 223 of the shroud 22 toward the upper inner circumferential surface 111 of the housing 10. A portion of the blood introduced through the suction inlet 14 passes through the space between the open vanes 24 to move to the space at the peripheral edge of the housing 10.

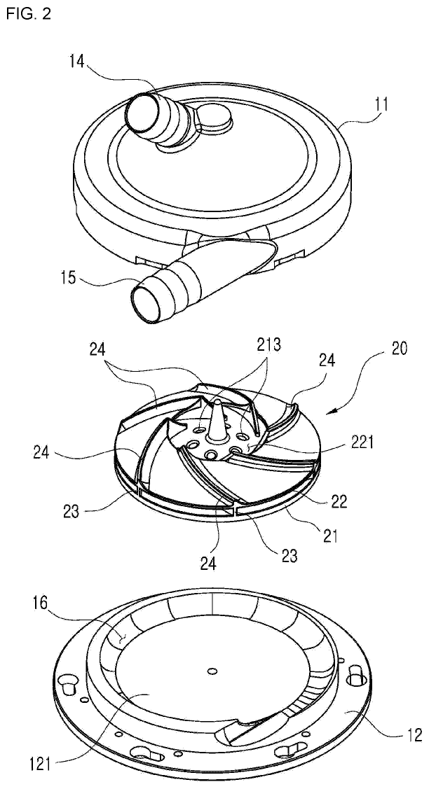

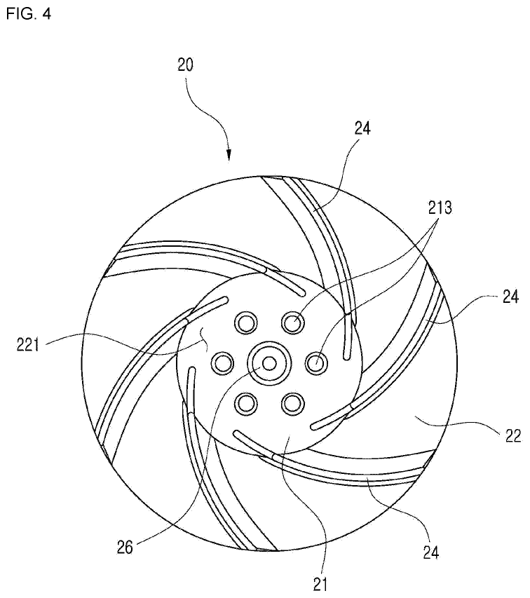

At this time, the shrouded vanes 23 and the open vanes 24 may be formed to be substantially vertically overlapped. For example, the shrouded vane 23 and the open vane 24 may have a curved shape. In FIG. 2 and FIG. 4, the rotation direction of the impeller 20 is a clockwise direction, and the vanes 23 and 24 may be formed such that the outer ends thereof are bent toward an opposite direction to the rotation direction of the impeller 20. The hemolysis phenomenon can be reduced by the bent shape of the vanes 23 and 24.

In addition, as shown in FIG. 2 and FIG. 4, the vanes 23 and 24 may not be formed in a region corresponding to the center area of the blood inlet hole 221 among the top surfaces of the base 21 and the shroud 22. Thereby, a stable inflow of blood can be achieved and a hemolysis phenomenon can be reduced.

Referring to FIG. 2, a radially inner end of the shrouded vane 23 may be formed such that an upper end thereof is located radially outside from a lower end thereof. For example, the inner end of the shrouded vane 23 may be formed to be inclined such that the upper end thereof is located radially outside from the lower end thereof. In addition, as shown in FIG. 2, the inner end of the shrouded vane 23 is not covered by the shroud 22, and the inclined line of the inner end may be connected to an inner upper end of the open vane 24 located thereabove. With this structure, hemolysis can be suppressed.

Meanwhile, in an embodiment of the present invention, it is configured to further reduce the hemolysis phenomenon through the shape of the outer ends of the vanes 23 and 24. Referring to FIG. 2, the outer end of the shroud vane 23 is formed to be vertical, and the outer end of the open vane 24 is formed such that a downstream side thereof in a rotation direction, i.e., a clockwise in FIG. 2, is inclined with respect to a vertical direction.

Further, according to an embodiment of the present invention, the ratio of the height of the outer end of the open vane 24 and the height of the outer end of the shrouded vane 23 is between 1:1 and 1:2.

The following table shows the results of computational fluid analysis using the numerical analysis program STAR-CCM+, where a is the height of the outer end of the open vane and b is the height of the outer end of the shrouded vane.

TABLE 1

| | descriptions | | | | | |
|---|---|---|---|---|---|---|
| | b = 0 | | | | | a = 0 |
| | (open | | | | | (shrouded |
| | impeller) | a:b = 1:1 | a:b = 1:1.5 | a:b = 1:2 | a:b = 1:3 | impeller) |
| flow rate(L/min) | 5 | 5 | 5 | 5 | 5 | 5 |
| rotation speed (rpm) | 400 | 400 | 400 | 400 | 400 | 400 |
| shear stress (max) [Pa] | 3518 | 3463 | 3435 | 3382 | 3360 | 3300 |
| volume of cell | 3.458 | 1.983 | 1.548 | 1.703 | 2.482 | 2.922 |
| Hemolysis [%] | 3.20 | 1.60 | 1.31 | 1.55 | 2.05 | 1.79 |

As shown in Table 1, it can be seen that hemolysis is relatively small when the ratio of the height of the outer end of the open vane 24 and the height of the outer end of the shrouded vane 23 is between 1:1 and 1:2. A blood circulation hole 213 connecting the bottom surface 211 and the top surface 212 of the base 21 may be formed. At this time, as shown in FIG. 3 and FIG. 4, the blood circulation hole 213 may be formed in a region where the vanes 23 and 24 are not formed. While the impeller 20 rotates, blood under the impeller 20 may move to the space above the impeller 20 through the blood circulation hole 213. The blood circulation hole 213 functions to circulate blood in the space below the impeller 20 to the upper space so as to prevent blood from accumulating and coagulating in the space below the impeller 20.

Meanwhile, the impeller 20 may be supported to the housing 10 in a double pivot structure via an upper pivot protrusion 26 and a lower pivot protrusion 25. Referring to FIG. 3, the upper pivot protrusion 26 and the lower pivot protrusion 25 protrude upward and downward, respectively, and may be supported on the upper inner circumferential surface 111 and the lower inner circumferential surface 121 of the housing 10, respectively. As shown in FIG. 3, the upper pivot protrusion 26 protrudes upward from the upper surface of the base 21 and is supported on the upper inner peripheral surface 111 of the housing 10. In this case, a pivot bearing 115 may be disposed at a region where the upper pivot protrusion 26 contacts. Meanwhile, as shown in FIG. 3, the lower pivot protrusion 25 protrudes downward and is supported on the lower inner peripheral surface 121 of the housing 10. In this case, a pivot bearing 215 may be disposed at a region where the lower pivot protrusion 25 contacts.

As shown in FIG. 3, the housing 10 forms a volute space 16 formed outside the radially outer end of the impeller 20. The volute space 16 extends circumferentially to surround the outer end of the impeller 20. For example, the volute space 16 may start from a point where the discharge outlet 15 is provided and may extend along the circumferential direction of the housing 10 to be connected to the discharge outlet. In this case, referring to FIG. 2, the volute space 16 may be formed such that its cross-sectional area gradually increases along the blood flow direction (clockwise in FIG. 4). This structure enables stable flow of blood and minimizes hemolysis.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

### INDUSTRIAL APPLICABILITY

The present invention relates to a centrifugal blood pump, so it has an industrial applicability.

1. A centrifugal blood pump comprising:
a housing;
a suction inlet for introducing blood into the housing;
an impeller that is rotatably disposed in the housing and imparts a centrifugal flow to the blood introduced through the suction inlet by rotation; and
a discharge outlet for discharging the blood given a centrifugal flow by the impeller,
wherein the impeller is formed in a double impeller structure including double vanes arranged vertically.

2. The centrifugal blood pump of claim 1, wherein the impeller comprises:
a base;
a shroud that is formed to be upwardly spaced apart from an upper surface of the base;
a shrouded vane extending between the upper surface of the base and a lower surface of the shroud; and
an open vane formed on an upper surface of the shroud.

3. The centrifugal blood pump of claim 2, wherein the shroud comprises an inlet hole that is formed on a center region thereof, and wherein the shrouded vane and the open vane are not formed on a center region of the base.

4. The centrifugal blood pump of claim 3, wherein the radially inner end of the shrouded vane is formed to be inclined such that an upper end thereof is located radially outside from a lower end thereof.

5. The centrifugal blood pump of claim 4, wherein an inner end of the shrouded vane is not covered by the shroud, and wherein an inclined line of the inner end of the shrouded vane is connected to an inner upper end of the open vane.

6. The centrifugal blood pump of claim 2, wherein an outer end of the shrouded vane is formed to be vertical, and wherein an outer end of the open vane is formed such that a downstream side thereof in a rotation direction is inclined with respect to a vertical direction.

7. The centrifugal blood pump of claim 1, wherein a ratio of a height of the outer end of the open vane and a height of the outer end of the shrouded vane is between 1:1 and 1:2.
